(19) United States
(12) Patent Application Publication
(10) Pub. No.: US 2016/0242448 A1
(43) Pub. Date: Aug. 25, 2016

(54) PROCESS FOR PROVIDING LUMINESCENCE IN OR FROM A FOOD PRODUCT

(71) Applicant: RUTGERS, THE STATE UNIVERSITY OF NEW JERSEY, New Brunswick, NJ (US)

(72) Inventors: Richard D. Ludescher, Somerset, NJ (US); Maria G. Corradini, Newark, NJ (US)

(21) Appl. No.: 15/028,257

(22) PCT Filed: Oct. 8, 2014
(86) PCT No.: PCT/US14/59777

§ 371 Date: Apr. 8, 2016

Related U.S. Application Data
(60) Provisional application No. 61/888,246, filed on Oct. 8, 2013.

(51) Int. Cl. A23L 1/27; A23G 3/34; C09K 11/06; A23G 3/36; A61K 9/20

(52) U.S. Cl. CPC A23L 1/2751; A23G 3/36; A23G 3/343; A23L 1/2753; A23L 1/2755; A23L 1/2756; A23L 1/2758; A61K 9/2072; C09K 11/06; A23V 2002/00

(57) ABSTRACT

Toe present invention relates to compositions containing photoluminescent substances in sufficient amount capable of emitting luminescence upon exposure to an external radiation source of stimulating photons. The present invention is also directed to methods of making and using such compositions. The use of such compositions can be with or without UV or visible light sources. Kit products containing such compositions' and light sources are also disclosed.

### CROSS-REFERENCE TO RELATED APPLICATIONS

This application claims priority under 35 U.S.C. §119(e) to U.S. Provisional Patent Application Ser. No. 61/888,246, filed on Oct. 8, 2013, which is hereby incorporated by reference in its entirety.

### FIELD OF THE INVENTION

The present invention relates to novel edible, safe, comestible compositions containing radiation energy absorbing substances capable of emitting luminescence (fluorescence or phosphorescence) upon exposure to an external radiation source. The present invention is also directed to methods of making and using such compositions.

### BACKGROUND OF THE INVENTION

Food products that either change color or glow in the dark have been described in the art. For example, US patent publication 20130017311 A1 provides for fluorescent candies and confections that glow under a black light. U.S. Pat. No. 6,120,821 describes food compositions having a color system with the ability to change its color when heated. U.S. Pat. No. 6,247,995 describes bioluminescent items that glow or produce or spew a bioluminescence. However, nowhere in the art describes the use of ingredients generally recognized as being safe as a source of generating luminescence.

Luminescence is the emission of light by a substance that is not resulting from heat. It can be caused by absorption of radiation (photoluminescence), chemical reactions, electrical energy, mechanical energy, and other reactions. Photoluminescent processes can be classified by various parameters including the energy of the exciting photon with respect to the emission. Bioluminescent products are those which emit luminescence when excited by chemical reaction mediated by a biological enzyme.

Although there have been reports on luminescent foods, they typically involve the use of a limited number of fluorescent food compounds such as chlorophyll, quinine and riboflavin. In such uses, the light source employed was generally a UV-A light, commonly known as “black light.” This particular light source emits long wave ultraviolet light (365 nm) that can be hazardous to consumers.

### SUMMARY OF THE INVENTION

The present invention describes novel compositions and methods of use thereof that were not previously described in the art.

The present invention relates to novel compositions comprising radiation energy absorbing substances in sufficient amount capable of generating an easily detected (by eye) signal upon exposure to an energy source. The present invention is also directed to methods of making and using such compositions. The signal generation can be accomplished by using a UV or a visible light source. Kits comprising such compositions and energy source, such as a light source are also disclosed.

In one aspect there is provided a composition comprising food, medicinal (e.g. pharmaceutical) products, drink, dietary supplement, or animal feed and one or more photoluminescent substances having the ability to emit luminescence upon exposure to a light source emitting photons with wavelengths that stimulate the substances. The light source provides photons having wavelengths in ranges from UV to visible light or infrared.

In some embodiments, the luminescence-generating substance is edible and prepared from a plant source including for example, fruit, vegetable, and grain. In some embodiments, the substance is a GRAS compound.

In some embodiments, the photoluminescence emitted is visible to the naked eye. In some embodiments, the luminescence-generating substance is disposed in the product in a way of selectively using the luminescent properties of the substance so that the photoluminescence generated therefrom comprises light having two or more wavelengths.

In another aspect of the invention, there is provided a method of providing photoluminescence from the above described composition by irradiating the composition with a radiation source emitting photons with wavelengths that stimulate the photoluminescent substances in the compositions until luminescence is emitted in or from the food product. In one embodiment the light sources emit photons with wavelengths ranging from 10 nm to 4000 nm In some embodiments, the emission wavelength range of the radiation source is 100-1000 nm. In some embodiments, the radiation can range from 200-800 nm. In some embodiments, the radiation is in the ranges of 250-550 nm. In some embodiments, the external radiation source emits photons at two or more wavelengths simultaneously or sequentially so that the photoluminescence emitted from the excited substance in the composition has a mixed color or changing color. The timespan and the temperature for exposing the substance is adjustable depending on the particular design of the substance and the composition.

In another aspect there is provided a method for determining authenticity, safety or integrity of a product by adding to the product a luminescence-generating substance, exposing the product to a light source emitting photons with a predetermined wavelength for a sufficient period of time to stimulate photoluminescence, and comparing any emission spectrum with a reference or standard spectrum to confirm the authenticity, safety or integrity of the product. Because the luminescence signals from the molecules in the food can be interpreted in terms of specific chemical and physical properties of the food (pH, viscosity, water content/activity, oxygen concentration, presence of metal ions, etc.), the method of the present invention can be applied to monitor and control food quality, shelf-life, and/or safety.

In another aspect there is provided a kit containing a luminescence-generating substance and a light source for emitting photons with wavelengths that stimulate the luminescence-generating substance. The luminescence-generating substance can be a GRAS compound or prepared from a plant source. The light source can be incorporated to various devices including a cooking appliance, a dishware, a container or glassware.

In another aspect there is provided a method of manufacturing a luminescence-emitting composition comprising a product and one or more substances in sufficient amounts capable of generating photoluminescence upon irradiation with photons having wavelengths that stimulate the luminescence-generating substances. In one embodiment, the substances generate photoluminescence upon exposure to photons with wavelengths between 100 nm and 1000 nm. The method of adding the substances to the product includes any sequence of mixing the two components to provide the composition.

### BRIEF DESCRIPTION OF THE DRAWINGS

FIG. 1 provides a picture of a dehydrated golden beet cake decoration.

FIG. 2 provides a list of food sources that contain at least one luminescent molecule. The table lists the photoluminescence, conditions, wavelengths and characteristics of the emitted light generated by each food source.

FIG. 3 provides for an array of LED lights that can be incorporated in dish and glassware.

### DETAILED DESCRIPTION OF THE INVENTION

A large number of colors, flavors or vitamins naturally found in foods or routinely added to food and pharmaceutical products can be fluorescent or phosphorescent under specific conditions and with appropriate excitation wavelengths. Conditions have been identified that cause luminescence to be generated in and from any products that contain such molecules. The present invention describes compositions comprising a sufficient amount of at least one photoluminescent substance capable of emitting photons upon exposure to an external energy source of photons with stimulating wavelengths.

While the following text may reference or exemplify specific compositions or methods, it is not intended to limit the scope of the invention to such particular reference or examples. Various modifications may be made by those skilled in the art, in view of practical and economic considerations, such as the source and characteristics of the luminescence-generating substances.

### Luminescence-Emitting Composition

In one aspect of the invention, there is provided compositions containing a processed product and one or more photoluminescent substances in sufficient amounts capable of generating photoluminescence upon irradiation by photons with stimulating wavelengths, typically between 100 nm and 1000 nm. The product refers to an article at any stage of its manufacture, including for example, processed raw material, manufacture of intermediate stage, and final products. The product can be a processed food product, drink, pharmaceutical product, dietary supplement, or animal feed.

In some embodiments, the product is prepared food product, which can be in the form of a liquid, a gelatinous material, a foam, a mousse, an emulsion, a semisolid, or a solid. Examples of various types of food products include liquids (e.g., soups, beverages, alcoholic beverages), viscous liquids (e.g., gravies, sauces), semi-solids (e.g., puddings), gels (e.g., gelatin), emulsions and foams (e.g., sauces, whipped cream, bread), and solids (e.g., sugar glass).

The composition contains one or more photoluminescent substances in a sufficient amount to emit luminescence upon sufficient exposure to an external radiation source of stimulating photons. In some embodiments, the concentration of the photoluminescent substance is between 0.001 to about 99.99% by weight of the composition, preferably in the range from 0.01 to about 20%, or more preferably in the range from 0.1 to 5%. In some embodiments, the disclosed food product comprises a protein, a fat, a carbohydrate, or combinations thereof.

The photoluminescent substance is preferably edible and can be readily prepared from a plant source. In some embodiments, the photoluminescent substance is prepared from a plant source including beets, turmeric root, yam, an orange, an apple, a pea, a rhubarb, a coconut, honey, maple syrup, refined and/or raw sugar, peanut butter, dulce de leche, a walnut, a berry, a mushroom, a bean, a pepper, or a chili or a combination thereof. In some embodiments, the photoluminescent substance from a plant source is in a liquid, solid, or semi-solid state. In some embodiments, the substance is dehydrated slices, alginate beads or is cooked. In some embodiments, the food source is fresh, intact-dehydrated, hydrated, or sauteed. In some embodiments, the photoluminescent component is a betaxanthin. In some embodiments, the betaxanthin is obtained from golden beets.

In some embodiment, the luminesce-generating substances include chlorophyll, riboflavin, vanillin, flavonols or flavins, betalains (betaxanthins, betacyanins), porphyrins, chlorophyll and other metalloporphyrins, erythrosin and similar synthetic food dyes, synthetic mono-azo and di-azo food dyes (sunset yellow, tartrazine, etc.), carotenoids, flavonoids, curcumin, anthocyanins and anthocyanidins, amino acids (tryptophan, tyrosine, phenylalanine), vitamins (retinol (A) and related molecules (retinal), thiamine (B₁), riboflavin (B₂) and its analogs, pyridoxine (B₆) and its metabolites, cyanocobalamin (B₁₂), calciferol (D₂), tocopherols (E) and folic acids, nucleic acid bases (pyrimidines and purines), alkaloids (quinine, caffeine, etc.), aromas and flavors that contain aromatic phenyl (vanillin, eugenol, etc.) or thiazole (2-acetyl thiazole, benzothiazole, etc.) or pyridine (2-acetyl pyridine, etc.) or pyrazine (acetyl pyrazine, trimethyl pyrazine, etc.) ring structures, various non-enzymatic and Maillard browning reaction products.

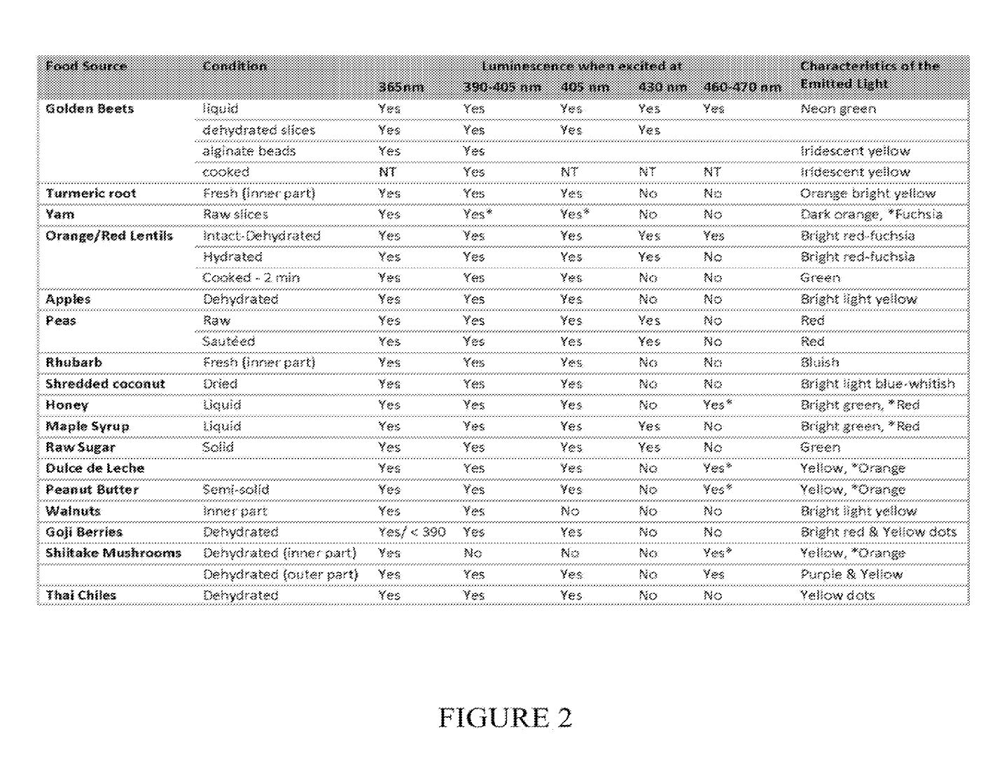

Multiple substances can be incorporated to a composition. In some embodiments, the composition contains at least 2, 3, 4, 5, 6, 7, or 8 photoluminescent substances. In some embodiments, the composition contains photoluminescent substances from at least 2, 3, 4, 5, 6, 7, or 8 of the above plant sources. The substance may be any of or a combination of the substances shown in FIG. 2.

The photoluminescent substance can be disposed in the food, animal feed or pharmaceutical compositions in such manner that it provides a pattern, a design, a sign or a message in, on or about the composition. In some embodiments, the photoluminescent substance is so arranged in the composition that the emitted light forms a pattern of image. The pattern can be any image including a colored shape or gradient, a character, or a figure. A pattern may have mixed color or changing color depending on the substances and the wavelengths of the exciting light. Because substances from various sources can illuminate at different wavelengths (see FIG. 2), the pattern may show up as same or different sequential images with same or different colors if the composition is exposed to light of different wavelengths sequentially. For example, depending on the condition (intact-dehydrated, hydrated, cooked) of orange/red lentils, the luminescence may vary from bright red to green. A composition containing both apple and orange (hydrated) may emit changing color from bright light yellow to bright red if the exciting wavelength shifts from 405 nm to 430 nm.

In some embodiments, the composition can be in the form of a bagel, a biscuit, a bread, a pancake, a waffle, a bun, a croissant, a sugar glass, a dumpling, a muffin, a refrigerated/frozen dough product, dough, baked beans, a burrito, chili, a taco, a tamale, a tortilla, a ready to eat cereal, a ready to eat meal, stuffing, a microwaveable meal, a brownie, a cake, a cheesecake, a coffee cake, a cookie, a dessert, a pastry, a sweet roll, a candy bar, a pie, a pie crust, pie filling, baby food, a baking mix, a batter, a breading, a gravy mix, a meat extender, a meat substitute, a seasoning mix, a soup or a soup mix, a gravy, a jello or gelatin, a salad dressing, a sour cream, a noodle, a pasta, noodles, an ice cream, a cracker, a doughnut, an egg roll, an extruded snack, a fruit and grain bar, a microwaveable snack product, a nutritional bar, a pretzel, a snack mix, a pizza or pizza crust, honey, peanut butter, a beverage, or animal food or pet food. In some embodiments, the food composition is gluten free.

In some embodiments, the invention provides compositions containing a pharmaceutical compound. In addition, the composition can be in the form of a liquid, a gelatinous material, a foam, an emulsion, a semisolid, or a solid form that contain at least one photoluminescent substance in a sufficient amount capable of generating a signal upon exposure to an external energy source and a pharmaceutically acceptable carrier. In some embodiments, the preferred amount of the photoluminescent substance can be in the range of 0.01 to 1% weight.

In some embodiments, the composition is a pharmaceutical composition that comprises a photoluminescent substance which emits luminescence when it is exposed to a light source that provides light ranging from UV to visible or to far-red wavelengths (200-800 nm). In some embodiments, the photoluminescent substance is obtained from a plant source. In another embodiment, the photoluminescent component is a GRAS component that emits luminescence upon exposure to a light source.

In some embodiments, the composition contains an additional component selected from the group consisting of vitamins, herbs, antioxidants, pH agents, chelators, diluents, viscosity modifiers, edible alkali metal salts, surfactants and any combinations thereof.

### Method for Providing Luminescence

In another aspect, the present invention provides a method for providing luminescence in or from a composition containing a product and one or more radiation energy absorbing substances capable of emitting luminescence. The method enhances or modifies the eating or drinking experience by providing luminescence emitting from an energy absorbing substance when the substance is exposed to a light source. The product and the luminescence-emitting substance in the composition are as described above. The method includes the steps of: (a) providing a composition comprising one or more photoluminescent substances in sufficient amounts capable of creating luminescence, (b) exposing said composition to an external radiation source emitting a wavelength with photons that stimulate the photoluminescent substance, typically ranging from 10 nm to 1000 nm, under conditions whereby luminescence is emitted in or from the composition. In at least one embodiment the optimum range of the radiation source is 100-800 nm. In another embodiment, the radiation can range from 200-800 nm, and in other embodiments the radiation is in the ranges of 250-750 nm, 300-750 nm, 350-750 nm, 400-750 nm, 450-750 nm, 50-750 nm, 350-700 nm, 400-700 nm, 450-700 nm, 500-700 nm.

The composition is exposed to the light source for a sufficient period of time to cause emission of luminescence from any part of the composition. In some embodiments, such period of time is at least 1 second. In some embodiments, the duration of time is at least 10, 20, 30, 40, 50, 60, 80, 120, 160, 200 or 600 seconds. In some embodiments, the exposure to light is performed in a cyclic pattern. In some embodiments, the duration of time is up to 1, 2 or 3 hours. In some embodiments, the composition includes a food product.

Various substances can be incorporated into the composition. In some embodiments of the present invention, the photoluminescent substance is designated by the U.S. Food and Drug Administration as Generally Recognized as Safe (GRAS). In some embodiments, the photoluminescent substance is a dietary component including beets, yam, an orange, an apple, a pea, a rhubarb, a coconut, honey, maple syrup, a walnut, a berry, a mushroom, a bean, a pepper, or a chili or a combination thereof.

In some embodiments, the substances that luminesce in foods include chlorophyll, riboflavin, vanillin, flavonols or flavins, betalains (betaxanthins, betacyanins), porphyrins, chlorophyll and other metalloporphyrins, erythrosin and similar synthetic food dyes, synthetic mono-azo and di-azo food dyes (sunset yellow, tartrazine, etc.), carotenoids, flavonoids, curcumin, anthocyanins and anthocyanidins, amino acids (tryptophan, tyrosine, phenylalanine), vitamins (retinol (A) and related molecules (retinal), thiamine (B₁), riboflavin (B₂) and its analogs, pyridoxine (B₆) and its metabolites, cyanocobalamin (B₁₂), calciferol (D₂), tocopherols (E) and folic acids, nucleic acid bases (pyrimidines and purines), alkaloids (quinine, caffeine, etc.), aromas and flavors that contain aromatic phenyl (vanillin, eugenol, etc.) or thiazole (2-acetyl thiazole, benzothiazole, etc.) or pyridine (2-acetyl pyridine, etc.) or pyrazine (acetyl pyrazine, trimethyl pyrazine, etc.) ring structures, various non-enzymatic and Maillard browning reaction products.

In some embodiments, the photoluminescent substance is selected from the group consisting of betalains (betaxanthins, betacyanins), porphyrins, chlorophyll and other metalloporphyrins, erythrosin and similar synthetic food dyes, synthetic mono-azo and di-azo food dyes (sunset yellow, tartrazine, etc.), carotenoids, flavonoids, curcumin, anthocyanins and anthocyanidins, amino acids (tryptophan, tyrosine, phenylalanine), vitamins (retinol (A) and related molecules (retinal), thiamine (B₁), riboflavin (B₂) and its analogs, pyridoxine (B₆) and its metabolites, cyanocobalamin (B₁₂), calciferol (D₂), tocopherols (E) and folic acids, nucleic acid bases (pyrimidines and purines), alkaloids (quinine, caffeine), aromas and flavors that contain aromatic phenyl (vanillin, eugenol, etc.) or thiazole (2-acetyl thiazole, benzothiazole, etc.) or pyridine (2-acetyl pyridine, etc.) or pyrazine (acetyl pyrazine, trimethyl pyrazine, etc.) ring structures, various non-enzymatic, and Maillard browning reaction products.

The types of devices that can be used to excite the luminescence include incandescent and compact fluorescent lamps, UV lamps, UV and visible light emitting diodes, lasers, automated light emission systems, etc. In some embodiments, such devices are incorporated into kitchen cookware, tableware, or other suitable consumer products. In some embodiments, consumer products include clothing, toys, safety products, sporting and camping goods such as diving sticks, plastic tubing, stationary, signs, and/or synthetic leathers.

The method of the present invention is preferably conducted at a temperature that does not interfere with the ability to excite the photoluminescent material. Such temperature can be in the range of 15 to 600° C. or 15 to 350° C. or 20 to 250° C. In a preferred embodiment, the temperature is in a range between 15 to 2000° C. In a more preferred embodiment, the temperature is 15, 16, 17, 18, 19, 20, 21, 22, 23, 24, 25, 26, 27, 28, 29, 30, 31, 32, 33, 34, 35° C. In the most preferred embodiment, the temperature is at refrigerated temperatures and/or ambient level.

In some embodiments, the product of the composition is a food product, which can be in the form of a liquid, a gelatinous material, a foam, a mousse, an emulsion, a semisolid, or a solid.

As described above, because substances from various plant sources can emit diverse sets of colors at different wavelengths (see FIG. 2), the luminescence pattern may show up as same or different sequential images with same or different colors if the food composition is exposed to light of different wavelengths is sequentially. Accordingly, the radiation source may provide light having different wavelengths at the same time or sequentially so that the substance of the composition generates a pre-designed luminescence.

In some embodiments, the composition contains food and can be in the form of a bagel, a biscuit, a bread, a pancake, a waffle, a bun, a croissant, a sugar glass, a dumpling, a muffin, a refrigerated/frozen dough product, dough, baked beans, a burrito, chili, a taco, a tamale, a tortilla, a ready to eat cereal, a ready to eat meal, stuffing, a microwaveable meal, a brownie, a cake, a cheesecake, a coffee cake, a cookie, a dessert, a pastry, a sweet roll, a candy bar, a pie, a pie crust, pie filling, baby food, a baking mix, a batter, a breading, a gravy mix, a meat extender, a meat substitute, a seasoning mix, a soup or a soup mix, a gravy, a jello or gelatin, a salad dressing, a sour cream, a noodle, a pasta, noodles, an ice cream, a cracker, a doughnut, an egg roll, an extruded snack, a fruit and grain bar, a microwaveable snack product, a nutritional bar, a pretzel, a snack mix, a pizza or pizza crust, honey, peanut butter, a beverage, or animal food or pet food. In another embodiment, the food composition is gluten free.

### Method for Detection

In another aspect of the invention, a method of determining authenticity, safety or integrity of a composition is described. A composition to be tested or examined can be products of various types, including for example, food, pharmaceuticals, dietary supplement, animal feed, processed raw materials, and an article at any stage of manufacture. Such method includes the steps: adding one or more of the photoluminescent substances of the present invention to a product to be authenticated, wherein said one or more of the substances are in sufficient amount to generate luminescence upon irradiation with sufficient stimulating infrared, visible or UV photons; exposing the product to a light source with stimulating photons of wavelengths ranging from UV to visible or far-red wavelengths for a time period sufficient to produce an emission spectrum and comparing the emission spectrum with a reference or standard spectrum to confirm the authenticity, safety or integrity of the product. Various changes in product integrity (e.g. dilution, adulteration, viscosity, pH, temperature) can be reflected in the emission spectrum. The luminescence-generating substance can be added to a product during the manufacturing process or prior to the testing of the product.

An emission spectrum can be in any digital or analog form to present detectable indicators. Non-limiting examples of the form of the emission spectra include an image, a color pattern or gradient, a data read, and a combination thereof.

Because the luminescence signals from an photoluminescent substance in a product can be interpreted in terms of specific chemical and physical properties of the product or its surrounding medium (pH, viscosity, water content/activity, oxygen concentration, presence of metal ions, temperature, light exposure, etc.), the photoluminescent substance serves as a probe and comparison of its emission spectrum with a standard or reference offers an efficient and convenient way to monitor or detect the quality, shelf-life, or safety of a product. Key detectable indicators include presence or absence of characteristic peaks at certain wavelengths, changes in photoluminescence intensity, and phosphorescence intensity and lifetime.

In exemplary embodiments, the method is used to detect the changes of viscosity in a liquid wherein an increase in viscosity restricts the molecular rotation of luminescence-emitting substances in the liquid and therefore leads to changes in photoluminescent intensity. In some embodiments, a change in photoluminescent intensity is indicative of exposure to elevated temperature beyond a desirable range due to sensitivity of the luminescence-emitting substance to the temperature of the surrounding medium. Exemplary temperatures of exposure that are detectable via luminescence emission spectra are 25, 30, 35, 40, 45, and 50° C. Exemplary increases of temperatures that are exposed to a product and are detectable via luminescence emission spectra are 5, 10, 15, and 20° C.

In exemplary embodiments, the detection method comprises: exposing a product containing one or more photoluminescent substances in sufficient amount capable of generating detectable luminescence upon stimulation with sufficient infrared, visible, or UV photons to a light source with suitable wavelengths and obtaining a reference/standard emission spectrum providing a product to be authenticated or tested containing said one or more photoluminescent substances; exposing the tested product to a light source with wavelengths that stimulate the photoluminescent substance; obtaining a report emission spectrum; and comparing key indicators in the both spectra from the reference product and the product to be tested for a difference in wavelength, color, or intensity.

Various types of photoluminescent substances, from natural source or synthetic means, can be incorporated into a product as a detection probe. Non-limiting examples of suitable substances include compound selected from the group consisting of betalains (betaxanthins, betacyanins), porphyrins, chlorophyll and other metalloporphyrins, erythrosin and similar synthetic food dyes, synthetic mono-azo and di-azo food dyes (sunset yellow, tartrazine, etc.), carotenoids, flavonoids, curcumin, anthocyanins and anthocyanidins, amino acids (tryptophan, tyrosine, phenylalanine), vitamins (retinol (A) and related molecules (retinal), thiamine (B₁), riboflavin (B₂) and its analogs, pyridoxine (B₆) and its metabolites, cyanocobalamin (B₁₂), calciferol (D₂), tocopherols (E) and folic acids, nucleic acid bases (pyrimidines and purines), alkaloids (quinine, caffeine, etc.), aromas and flavors that contain aromatic phenyl (vanillin, eugenol, etc.) or thiazole (2-acetyl thiazole, benzothiazole, etc.) or pyridine (2-acetyl pyridine, etc.) or pyrazine (acetyl pyrazine, trimethyl pyrazine, etc.) ring structures, various non-enzymatic and Maillard browning reaction products, Citrus Red, Allura Red, Sunset Yellow and Fast Green.

In some embodiments, the photoluminescent substances are prepared from a natural source including a beet, turmeric root, yam, an orange, an apple, a pea, a rhubarb, a coconut, honey, maple syrup, refined and/or raw sugar, dulce de leche, peanut butter, a walnut, a berry, a mushroom, a bean, a pepper, or a chili or a combination thereof.

The present invention also provides for methods of identification or detection utilizing compositions containing photoluminescent GRAS materials whose emission signature lies partly or fully in the UV to visible light region of the electromagnetic spectrum. GRAS molecules that naturally occur in or are routinely added to foods (colors, flavors, etc.) can be used as intrinsic and safe luminescent probes of important chemical and physical properties in food or other products. Such molecules can provide information about local pH, solvent polarity, oxygen concentration (and diffusion), solution viscosity, local structural organization, and other properties, and thus can be used to monitor food quality, stability and bioavailability at every stage of the production and distribution chain from evaluation of raw materials at the production plant to final consumption in a restaurant, food service facility, or at home.

The amount and ratio of one or more photoluminescent substances added to the product depends on factors such as specific characteristics and function of each substance, the product to be tested, and the surrounding medium and can be readily determined by one of ordinary skill in the art without undue experiments. In exemplary embodiments, at least 2, at least 3, or at least 4 luminescence generating substances are added to the product.

Photoluminescence spectra of a luminescence-generating substance can be affected by various factors of the surrounding medium including pH, temperature, salt concentration, and solvent. For example, for substances of 3-hydroxy flavone (HF) structure type, the presence of metal salt (e.g. iron and calcium) may result in formation of a metal-3HF complex which will in turn change characteristics of the photoluminescence spectrum such as intensity and wavelength of the peaks. The effect can also be modulated by adjusting the concentration of the salt solution. Other examples of metals include salts of sodium, potassium, magnesium, and combination thereof. Therefore, certain photoluminescence spectra are indicative of the presence of particular metal cations in a corresponding range of concentration. Alternatively, the addition of a metal salt may enhance the sensitivity of a Photoluminescence spectrum in the detection of a product. Further, solvents with different polarity or hydrogen-bonding capacity will also impact the spectrum of a substance, for example, by affecting the coordination between molecules of the substances and surrounding metal cations.

Viscosity of the surrounding medium also affects the photoluminescence emission of certain photoluminescent substances. For substance with emission spectra produced by molecular rotation, increases in viscosity confine the ability of the molecules to rotate and change the emission spectra. When placed in a low viscosity medium, molecules of the substance undergo fast internal rotation in the excited state, and thus fast radiationless decay that quenches photoluminescence. Conversely, environmental restrictions to twisting in the excited state due to high viscosity environment can cause a dramatic increase in the photoluminescence emission intensity of the probe composition. For example, in solutions of glycerol, glycerol-ethylene glycol, glycerol-water or sucrose, probes comprising Citrus Red, Allura Red, Sunset Yellow or Fast Green exhibit a marked sensitivity to the rheological properties of the medium, e.g., as the viscosity increased, the photoluminescence intensity increased. The molecular weight of a thickening agent (e.g. hydrocolloid) in the product to be tested, its degree of polymerization and the affinity of the probe for the thickening agent also affect the photophysical response. The present invention utilizes all these characteristics to monitor rheological properties of a product or a medium with the luminescence generating substances as an intrinsic sensor.

The presence of ordinary or reversed micelles in surrounding medium has the effect of enhancing the photoluminescence intensity or causing peak-shifting in the spectrum of a luminescence generating substances. For example, the maximum photo-luminescence intensity of all fluorescent emissions for anthocyanins and betalains micellar (betacyanins and betaxanthins) increases at least two-fold when the substances are in a surrounding medium with anionic micelles. Such change can be explained by the rationale that anthocyanins and betalains exhibit molecular rotor behavior in which internal rotational motion quenches the excited state, using anionic micelles in the surrounding medium results in decrease on the rate of rotational mobility of the main segments of the fluorophores due to counter-ion binding between the positively charged pigments and the anionic surfactant aggregates.

Temperature may also affect the phosphorescence spectra of the probe composition. For example, a composition including riboflavin when being exposed to temperatures above 30° C. shows a marked decrease in phosphorescence intensity and lifetime. Such a decrease in photophysical properties makes riboflavin a suitable sensor for temperature abuse.

Methods of the present invention can be applied to the detection of various conditions (e.g. presence of certain cations, viscosity, pH, solvent polarity) of a product or a medium. Meanwhile, various medium factors including pH modifiers, salts, suitable solvents, ordinary or reversed micelles, or other auxiliary agents may also be added to a product to enhance or facilitate the detection of photoluminescent spectra from luminescence generating substances in the product. The amount and ratio of different medium factors of the probe composition depends on factors such as specific characteristics and function of each medium factor and the product to be tested and can be readily determined by one of ordinary skill in the art without undue experimentation.

The method can also be applied to the quality monitoring and control of pharmaceutical products. Currently pills are identified by their imprint, size, shape, or color. These features allow for verification of doses and in some cases for identification of counterfeits. In some embodiments, the photoluminescent substance can be a part of a drug formulation. The luminescence-generating substance can be added to a pharmaceutical product during any stage of its manufacturing process, including for example, after production of API, during formulation, and before packaging. According to this aspect of the invention, for example a suitable fluorescent compound, such as riboflavin, can be incorporated into the drug formulation. In some embodiments, luminescence spectroscopy can be used to detect a counterfeit version of the drug formulation. In some embodiments, a luminescent pattern made from a compound in the drug can be used to identify a counterfeit product. In some embodiments, other known luminescence techniques may be employed to identify changes in the drug formulation.

The present invention is further directed to the incorporation of the photolunminescent substances of the present invention as GRAS probes into the pills as markers of dosage, source, and other identifiers, and allow for control of shelf life and detection of mishandling or mispackaging or incorrect dispensing. The applications thus include use as indicators of the type or dose of the drug as well as indicators of shelf-life, spoilage or tampering. In some embodiments, the present invention can be used to assess authenticity of the pharmaceutical composition using the GRAS probes, in the same manner as using non-GRAS probes. The GRAS probes can be selected based on the sensitivity of the luminescent properties to known factors that affect the stability of the active component (the drug of interest), such as Oz exposure or relative humidity. The GRAS luminescent probes of the present invention provide identification and also act as a quality tag whose photophysical properties can be easily assessed using hand held devices.

### Kit for Providing Luminescence

In another aspect of the invention, a kit is disclosed that comprises one or more photoluminescent substances capable of generating luminescence and an energy source. The substances are in a sufficient amount capable of creating luminescence. The energy source is a device that produces light at preselected wavelengths ranging from 100 nm to 1,000 nm capable of exciting the photoluminescent component to emit luminescence. The luminescence-generating substance can be added to a product (e.g. a food, dietary supplement, pharmaceutical product). The substance emits luminescence upon being exposed to a light source with suitable wavelength. In some embodiments, the kit further includes a food product, a diet supplement, or a pharmaceutical product.

In some embodiments, the energy absorbing substance can be selected from a food source including beet, turmeric root, yam, orange, apple, pea, rhubarb, coconut, honey, maple syrup, refined and raw sugar, dulce de leche, peanut butter, walnut, berry, mushroom, bean, pepper, and chili, and a combination thereof.

In some embodiments, the energy absorbing substance is selected from betaxanthins, betacyanins, porphyrins, chlorophyll metalloporphyrins, erythrosin and synthetic analogs thereof, synthetic mono-azo and di-azo food dyes, carotenoids, flavonoids, curcumin, anthocyanins, anthocyanidins, amino acids, vitamins, retinol (A) and analogs thereof, thiamine (B₁), riboflavin (B₂) and analogs thereof, pyridoxine (B₆) and metabolites thereof, cyanocobalamin (B₁₂), calciferol (D₂), tocopherols (E), folic acids, nucleic acid bases, alkaloids, phenyl-containing aromas and flavors, vanillin, eugenol, thiazole, 2-acetyl thiazole, benzothiazole, substituted or unsubstituted pyridine, 2-acetyl pyridine, substituted or unsubstituted pyrazine, acetyl pyrazine, trimethyl pyrazine, non-enzymatic browning reaction products, Maillard reaction products, and a combination thereof. In some embodiments, the energy absorbing substance is a GRAS compound.

Those of ordinary skill in the art would appreciate that the present inventors disclose substance-specific conditions that would allow a food product to exhibit luminescence. For example by selecting specific wavelengths and physical state, an energy absorbing substance obtained from plant sources such as yam, honey, Dulce de Leche, peanut butter, shiitake mushrooms, luminescence is generated with appropriate excitation and the color of the emitted light can be controlled. FIG. 2 provide a list of substances, their respective luminescent wavelengths and the characteristics of the light emitted from the substances.

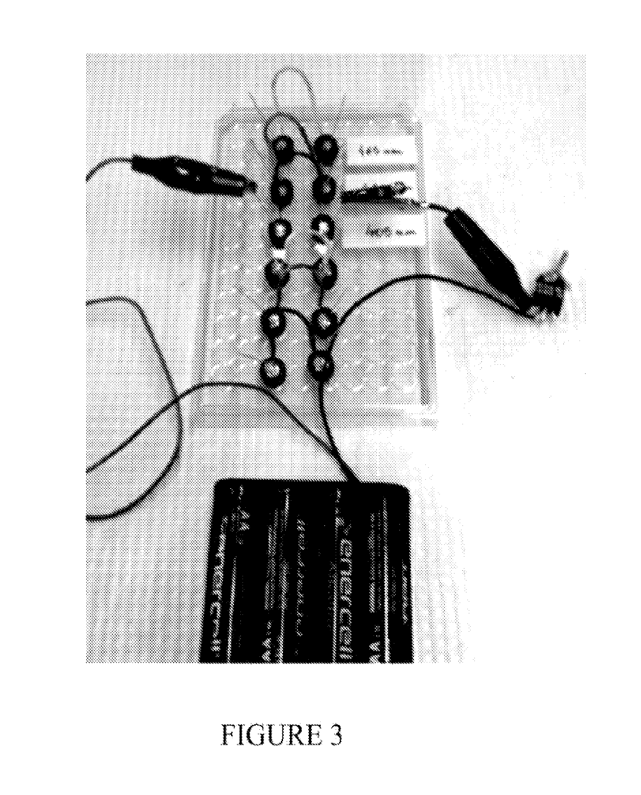

Devices of the kit of the present invention deliver light of the appropriate wavelength, in the appropriate geometry to excite the luminescence in specific substances. In some embodiments, the generated light is presented in “glow in the dark” foods to the consumer. FIG. 3 depicts an array of LED lights that can be incorporated in dish and glassware.

The types of devices that can be used to excite the luminescence include incandescent and compact fluorescent lamps, UV lamps, UV and visible light emitting diodes, lasers, automated light emission systems, etc. In some embodiments, such devices are incorporated into kitchen cookware, tableware, or other suitable consumer products.

Light at preselected wavelengths can be produced from the device simultaneously or in a pre-designed sequence. In some embodiments, the light source produces wavelengths in the ranges of 150 nm to 800 nm, 200 nm to 750 nm, 300 nm to 700 nm, 350 nm to 450 nm, 350 nm to 475 nm, 350 nm to 500 nm, 400 am to 700 nm, 450 nm to 700 nm, 450 nm to 650 nm, or 550 nm to 700 nm. Exemplary wavelengths of light produced from the light source include 365 nm, 390 nm, 395 nm, 400 nm, 405 nm, 390-405 nm, 410 nm, 415 nm, 420 nm, 425 nm, 410-425 nm, 430 nm, 435 nm, 440 nm, 445 nm, 450 nm, 455 nm, 430-455 nm, 460 nm, 465 nm, 470 nm, 460-470 nm, 475 nm, 480 nm, 485 nm, 490 nm, 495 nm, and 500 nm. In some embodiments, the light source is a UV source producing UV light having a wavelength of from 100 nm to 400 nm. In some embodiments, the visible or UV light source is an LED source that can be incorporated into cookware, a dish, a container or glassware.

The photoluminescent substance of the kit can be disposed within a product (e.g. food, dietary supplement, and pharmaceutical) in a pattern and exposure of the substance to radiation for a sufficient period of time generates luminescence. Such period of time can range from less than 1 second to 3 hours.

The foods that can be made luminescent by the kit of the present invention include liquids (e.g., soups, beverages, alcoholic beverages), viscous liquids (e.g., gravies, sauces), semi-solids (e.g., puddings), gels (e.g., gelatin), emulsions and foams (e.g., sauces, whipped cream, bread), and solids (e.g., sugar glass).

The food can be one of a bagel, a biscuit, a bread, a pancake, a waffle, a bun, a croissant, a sugar glass, a dumpling, a muffin, a refrigerated/frozen dough products, dough, baked beans, a burrito, chili, a taco, a tamale, a tortilla, a ready to eat cereal, a ready to eat meal, stuffing, a microwaveable meal, a brownie, a cake, a cheesecake, a coffee cake, a cookie, a dessert, a pastry, a sweet roll, a candy bar, a pie, a pie crust, pie filling, baby food, a baking mix, a batter, a breading, a gravy mix, a meat extender, a meat substitute, a seasoning mix, a soup or a soup mix, a gravy, a jello or gelatin, a salad dressing, a sour cream, a noodle, a pasta, noodles, an ice cream, a cracker, a doughnut, an egg roll, an extruded snack, a fruit and grain bar, a microwaveable snack product, a nutritional bar, a pretzel, a snack mix, a pizza or pizza crust, honey, peanut butter, a beverage, or animal food or pet food.

### Method of Preparation

In another aspect there is provided a method of preparing a luminescence-emitting composition, comprising adding to a product one or more photoluminescent substances in sufficient amounts capable of generating photoluminescence upon stimulation with photons having wavelength between 100 nm and 1000 nm. The product can be a food product, drink, pharmaceutical product, dietary supplement, or animal feed.

The photoluminescent substance is added to the product in a manner so that the resulting composition emit a pre-designed luminescence upon stimulation with sufficient infrared, visible or UV photons with stimulating wavelengths. The amount and ratio of the substances to be added depend on the design of the luminescence and the characteristics of the substance and can be readily determined by one of ordinary skill in the art without undue experimentation. In exemplary embodiments, at least 2, 3, 4, 5, or 6 luminescence-generating substances are added to a product to prepare a luminescence-emitting composition.

In some embodiments, the luminescence-generating substance is edible and prepared from a plant source selected from the group consisting of beet, turmeric root, yam, orange, apple, pea, rhubarb, coconut, honey, maple syrup, refined and raw sugar, dulce de leche, peanut butter, walnut, berry, mushroom, bean, pepper, and chili.

In some embodiment, the luminescence-generating substance is selected from betaxanthins, betacyanins, porphyrins, chlorophyll metalloporphyrins, erythrosin and synthetic analogs thereof, synthetic mono-azo and di-azo food dyes, carotenoids, flavonoids, curcumin, anthocyanins, anthocyanidins, amino acids, vitamin, retinol (A) and analogs thereof, thiamine (B₁), riboflavin (B₂) and analogs thereof, pyridoxine (B₆) and metabolites thereof, cyanocobalamin (B₁₂), calciferol (D₂), tocopherols (E), folic acids, nucleic acid bases, alkaloids, phenyl-containing aromas and flavors, vanillin, eugenol, thiazole, 2-acetyl thiazole, benzothiazole, substituted or unsubstituted pyridine, 2-acetyl pyridine, substituted or unsubstituted pyrazine, acetyl pyrazine, trimethyl pyrazine, non-enzymatic browning reaction products, and Maillard reaction products. In some embodiment, the substance is a GRAS compound.

### Example

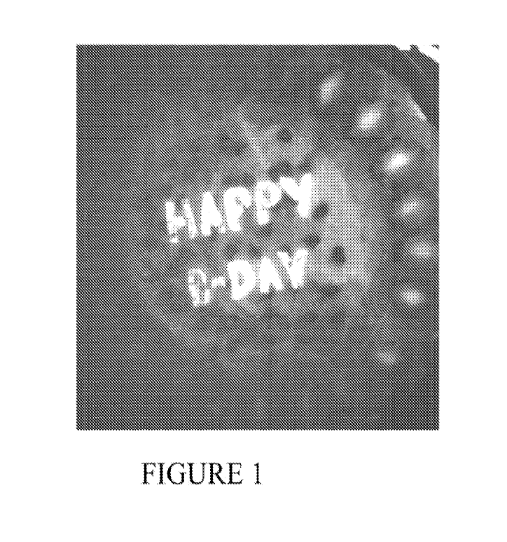

Dehydrated golden beets were carved as letters and were placed on top of a previously baked cake. FIG. 1 shows the design emitted from the cake under appropriate excitation.

### OTHER APPLICATIONS

The techniques described in this application are applicable to any material that is consumed by humans or by other animals. As described above, the present invention can be used to ascertain the quality and shelf-life and to ensure the safety of pet and animal foods under conditions from production to consumption.

These techniques are also applicable for non-food consumer products, such as toothpaste, mouthwash, cosmetics, etc., that are used under conditions where they might be consumed or enter the digestive system inadvertently, or that are used in such a fashion that the physical and chemical state or microbiological safety of the product is of concern and the remedy for these concerns is enhanced by the use of GRAS, or “natural” components. They are thus fully applicable for monitoring the integrity, the authenticity and even the degree of microbial contamination of any such products

Those skilled in the art will appreciate that they can readily use the disclosed conception and specific embodiments as a basis for designing or modifying other structures and identifying other luminescence compounds or foods for carrying out the same purposes of the present invention and that such other structures do not depart from the spirit and scope of the invention in its broadest form.

1. A composition comprising a processed product and one or more photoluminescent substances in sufficient amounts capable of generating photoluminescence upon irradiation with photons with stimulating wavelengths, wherein said one or more photoluminescent substances are edible and prepared from a plant source.

2. The composition of claim 1, wherein the processed product is a food product, drink, pharmaceutical product, dietary supplement, or animal feed.

3. The composition of claim 1, wherein said one or more photoluminescent substances are selected from the group consisting of beet, turmeric root, yam, orange, apple, pea, rhubarb, coconut, honey, maple syrup, refined and raw sugar, peanut butter, walnut, berry, mushroom, bean, pepper, and chili.

4. The composition of claim 1, further comprising one or more photoluminescent substances are selected from the group consisting of betaxanthins, betacyanins, porphyrins, chlorophyll metalloporphyrins, erythrosin and synthetic analogs thereof, synthetic mono-azo and di-azo food dyes, synthetic tri aryl methane dyes, carotenoids, flavonoids, curcumin, anthocyanins, anthocyanidins, amino acids, vitamins, retinol (A) and analogs thereof, thiamine (B₁), riboflavin (B₂) and analogs thereof, pyridoxine (B₆) and metabolites thereof, cyanocobalamin (B₁₂), calciferol (D₂), tocopherols (E), folic acids, nucleic acid bases, alkaloids, phenyl-containing aromas and flavors, vanillin, eugenol, thiazole, 2-acetyl thiazole, benzothiazole, substituted or unsubstituted pyridine, 2-acetyl pyridine, substituted or unsubstituted pyrazine, acetyl pyrazine, trimethyl pyrazine, non-enzymatic browning reaction products, and Maillard reaction products.

5. The composition of claim 1, wherein said one or more photoluminescent substances are compounds generally recognized as safe (GRAS).

6. The composition of claim 1, wherein the photoluminescence generated from said one or more substances is visible to the naked eye.

7. The composition of claim 1, wherein the photoluminescence has a pre-designed color pattern or intensity.

8. The composition of claim 1, wherein said one or more photoluminescent substances are disposed in the composition in such a manner that the photoluminescence generated therefrom comprises light having two or more wavelengths.

9. The composition of claim 1, further comprising an additional component selected from the group consisting of vitamins, herbs, antioxidants, pH regulators, chelators, diluents, viscosity modifiers, edible alkali metal salts, surfactants and any combinations thereof.

10. The composition of claim 1, wherein the product is a pharmaceutical product.

11. The composition of claim 10, wherein the pharmaceutical product is in the form of liquid, gelatinous material, foam, emulsion, semisolid, or solid.

12. A method for providing photoluminescence in or from a composition comprising the steps of:
(a) providing the composition of claim 1; and
(b) exposing the composition to an external radiation source of photons with stimulating wavelengths under conditions whereby photoluminescence is emitted in or from the composition.

13. The method of claim 12, wherein the external radiation source of step (b) emits photons at two or more stimulating wavelengths simultaneously or sequentially so that the photoluminescence emitted from the composition has a mixed color or changing color.

14-17. (canceled)

18. A method for determining the authenticity, safety or integrity of a product comprising the steps:
(a). adding one or more photoluminescent substances to said product, wherein said one or more substances are in sufficient amount capable of generating luminescence upon irradiation with sufficient stimulating infrared, visible or UV photons, and wherein said one or more photoluminescent substances are edible and prepared from a plant source;
(b). exposing the product to a light source that provides light ranging from UV to visible or far-red wavelengths with stimulating photons to produce an emission spectrum and comparing the emission spectrum with a reference or standard spectrum to confirm the authenticity, safety or integrity of the product.

19-37. (canceled)

38. The composition of claim 1, wherein said stimulating wavelengths are selected from the group consisting of 365 nm, 390-405 nm, 405 nm, 430 nm, 460-470 nm and 500-600 nm.

39. The composition of claim 1, wherein said one or more photoluminescent substances are a solid, a semi-solid or a liquid.

40. The composition of claim 1, wherein said one or more photoluminescent substances are dehydrated, cooked, emulsified, or sauteed, processed into alginate beads, or included into a formulation to generate a desirable photoluminescence upon irradiation.

41. The composition of claim 1, comprising two or more photoluminescent substances, said two or more photoluminescent substances generate photoluminescence of different colors upon irradiation at the same stimulating wavelengths.

42. The composition of claim 1, comprising two or more photoluminescent substances, said two or more photoluminescent substances generate photoluminescence of different colors upon irradiation at different stimulating wavelengths.

43. The composition of claim 1, wherein said one or more photoluminescent substances are disposed in the composition in such a manner that the photoluminescence generated therefrom forms an image selected from the group consisting of a shape, a color gradient, a character, and a figure.
